(19) United States
(12) Patent Application Publication
(10) Pub. No.: US 2003/0018343 A1
(43) Pub. Date: Jan. 23, 2003

(54) Medical device delivery system having reduced loading, and method

(76) Inventor: Mark L. Mathis, Kirkland, WA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/910,975

(22) Filed: Jul. 23, 2001

(51) Int. Cl. A61F 11/00

(52) U.S. Cl. 606/108; 623/1.11; 623/1.19

(57) ABSTRACT

The present invention involves medical devices, and also the delivery systems used to convey them to a desired location for treatment and then deploy them in position. Of course, it is desirable for many reasons to reduce the amount of stress or loading in the compressed medical device. Less stress generally means the device is more flexible during delivery, less friction during deployment, less possibility of device failure. Also, less stress may indicate the delivery system can obtain a longer shelf life, needs to support less expansive force, and can be designed with greater flexibility and smaller dimensions to reach smaller and more delicate anatomy. The novel technique of the present invention includes intentionally “over-compressing” the stent or other medical device to a size slightly smaller than eventually desired, using much greater pressure, and then “relaxing” the stent by allowing it to expand slightly to the desired initial size. The result of this over-compress and relax process is to radically reduce the stress in, and the expansive force of the stent against, the delivery system.

### BACKGROUND AND SUMMARY OF THE INVENTION

1. Technical Background

The present invention relates generally to medical devices, and more particularly to a medical device delivery system having a reduced loading configuration.

2. Discussion

The present invention involves medical devices, and also the delivery systems used to convey them to a desired location for treatment and then deploy them in position. Many such devices are initially compressed to a smaller size for storage and delivery, and then resiliently expand to a larger deployed size. As a result, the medical device may be constantly trying to expand during storage and delivery, and is often held compressed to the desired relatively small initial size by some component of the delivery system.

With prior resilient medical devices and delivery systems, this expansion force may be maximized to enhance the strength of the deployed medical device. Accordingly, the delivery system may be designed to support the constant stress of a relatively large expansion force throughout sterilization, storage, device delivery and deployment.

Of course, it is desirable for many reasons to reduce the amount of stress or loading in the compressed medical device. Less stress generally means the device is more flexible during delivery, less friction during deployment, less possibility of device failure. Also, less stress may indicate the delivery system can obtain a longer shelf life, needs to support less expansive force, and can be designed with greater flexibility and smaller dimensions to reach smaller and more delicate anatomy.

Many prior delivery systems take the form of vascular catheters used in a variety of therapeutic applications. In some types of vascular disease, blood vessels may be partially or totally blocked or narrowed by a lesion or stenosis, and may take the form of hard plaque, cholesterol, fats, or viscous thrombus. Such a stenosis may cause heart attack or stroke, which are significant health problems affecting millions of people each year. Typical disease patterns involve stenosis development, causing a blockage or partial blockage at the site.

For example, various procedures are well known for addressing stenoses and opening body vessels that have a constriction due to plaque buildup or thrombus, etc. With such procedures, an expansive force may be applied to the lumen of the stenosis. This outward pressing of a constriction or narrowing at the desired site in a body passage is intended to partially or completely re-open or dilate that body passageway or lumen, increasing its inner diameter or cross-sectional area. The objective of this procedure is to increase the inner diameter or cross-sectional area of the vessel passage or lumen through which blood flows, to encourage greater blood flow through the newly expanded vessel.

Often, it is deemed to be desirable to leave a medical device in place at the site, to provide continuing support for the vessel wall at that location. Such a device may provide a scaffold type of structure for example, which tends to hold open the vessel. Endothelium development may be allowed to occur to help repair the diseased, injured or damaged area. This scaffold device tends to hold the lumen open longer, to reinforce the vessel wall and improve blood flow. It is referred to as a “stent” or endoprosthesis, and may have various designs, often with a resilient, flexible and cylindrical spring shape. In some cases, the stent is a flexible cylinder or scaffold made of metal or polymers which may be permanently implanted into the vessel.

Stenting has come to be an accepted interventional medical procedure in many situations where vessels require support on a long-term basis. In operation, a catheter is used to transport the stent into and through a blood vessel, until the stent or the like is positioned at a desired location. Once at the desired location, the stent is deployed to provide internal support of the vessel or other treatment.

Some stents are deployed by an angioplasty balloon catheter, and are referred to as balloon-expandable stents. Another type of stent is of the self-expanding variety. Self-expanding stents tend to resiliently expand from an initial diameter, and must be held constantly in compression during delivery to remain at the initial diameter. Typically, a cylindrical sheath or similar device over the stent is needed to compress and hold the stent, endoprosthesis, or other resilient medical device at a small initial diameter during delivery and passage through the body vessel. Once the treatment site is reached, the sheath is withdrawn from around the stent, and the stent resiliently expands in place of its own accord.

This invention generally relates to resilient medical devices, including stents of the self-expanding type. More particularly, the invention relates to self-expanding stents or other resilient medical device delivered in a small initial size under radial compression, which are deployed by releasing a restraining member so the medical device resiliently expands at that location.

The example case where a stent is selected as the resilient medical device, the stent may be made of a resilient and preferably metal tube, with cuts or slits removed to form a lattice. Examples of this kind of “slotted tube” stent design are illustrated in FIGS. 4-8. The stent may also be formed of a continuous wire strand shaped into a generally cylindrical member and having a plurality of coiling and/or undulating spring portions wound from the strand so as to impart the desired radial strength. An example of this kind of “wire-wound” stent design is illustrated in FIG. 9.

Purely as an example of a possible therapeutic application, the present invention will be described in relation to coronary, peripheral, and neurological vascular stents. The coronary procedure is often referred to as “coronary stenting.” However, it should be understood that the present invention relates to any resilient medical device delivery system having the reduced loading features of the present invention, and is not limited to a particular device design, or a particular deployment system or location. Indeed, the techniques of the present invention are readily applicable to other resilient medical devices including vascular filters, vascular grafts, valves, clips such as anastomosis and aneurysm clips, coaxial tube devices such as endoscopes and optical fiber guides, and orthopedic devices such as bone anchors and surgical devices.

Some catheters have a relatively long and flexible tubular shaft defining one or more passages or lumens, and may deliver and deploy a stent near one end of the shaft. This end of the catheter where the stent is located is customarily referred to as the “distal” end, while the other end is called the “proximal” end. The proximal end of the shaft may lead to a hub coupling for connecting the shaft and the lumens to various equipment. Examples of stents and catheters are shown in U.S. Pat. No. 6,019,778 issued to Wilson et al., on Feb. 1, 2000 entitled “Delivery Apparatus For A Self-Expanding Stent,” which describes a self-expanding stent delivery system.

A common treatment method for using such a catheter is to advance the catheter into the body of a patient, by directing the catheter distal end percutaneously through an incision and along a body passage until the stent is located within the desired site. The term “desired site” refers to the location in the patient's body currently selected for treatment by a physician. After the stent is deployed within the desired site, it will tend to resiliently expand to hold the body passage open.

It is of course desirable to retain the stent securely in the proper position, and the delivery system should also preferably protect the stent from damage or deformation during delivery. The delivery system is also preferably flexible and able to push through and traverse as many different anatomical arrangements and stenosis configurations as possible. Moreover, the delivery system should preferably have a positive mechanism for holding, and then selectively releasing and deploying the stent at the desired site. The delivery system also preferably includes a mechanism for securing the stent in the form of a sheath, capable of completely covering and compressing the stent during insertion.

The novel medical device delivery system and method of the present invention relates to any resilient medical device having physical properties characterized by a stress-strain curve generally similar to the sample diagram in FIG. 2. The shape of this diagram is called a “hysteresis loop,” because the stress of the device is different depending on whether it is being compressed or released. An example of such a medical device is the resilient self-expanding vascular stent, which may often take the form of a cylindrical mesh tube. Self-expanding stents may be made of an elastic material, or a “super-elastic” material such as nitinol, which is a metal alloy of nickel and titanium.

Medical device delivery systems are often designed for the smallest possible outer diameter or “profile” at the distal end. This small profile may be preferred for access into small vessels following balloon angioplasty, or during a procedure called “direct stenting” to open a vessel without first performing angioplasty. Also, small profiles generally have the benefits of small access sites, and less possibility of trauma or vessel spasm.

Accordingly, the present invention may provide a delivery system for delivering and deploying a resiliently expandable medical device, including for example a stent. Because of the shape of the stress-strain diagram for the resilient medical device, the stress in the material is greatest when it is compressed during assembly into the delivery system. As a result, the delivery system has been generally designed to support this high stress throughout the stocking and distribution process, which often includes heat and sterilization, or months of stocking delays. The delivery system is thus required to be larger, with thicker components, and is generally stiffer than necessary.

The novel technique of the present invention includes intentionally “over-compressing” the stent or other medical device to a size slightly smaller than eventually desired, using much greater pressure, and then “relaxing” the stent by allowing it to expand slightly to the desired initial size. The result of this over-compress and relax process is to radically reduce the stress in, and the expansive force of the stent against, the delivery system.

Indeed, the stress present in the stent or other medical device may be reduced by as much as a factor of magnitude. The delivery system can accordingly be designed with a smaller distal profile, thinner components, and have a longer shelf life and greater flexibility, because it needs to support and contain less stress or expansive force.

The reason for this greatly reduced stress can be understood graphically by referring to FIG. 2. When the stent or other resilient medical device is first made, it has an initial expanded and relaxed size, at position A in FIG. 2. When it is compressed to the smaller size necessary to fit in the delivery system, at approximately position B, it presses outward with high stress. Prior devices and delivery systems carry this relatively higher stress level throughout packaging, sterlization, storage, shipping, and delivery into the patient.

However, according to the present invention, the device is over-compressed approximately to position C in FIG. 2, at slightly higher stress and compressed to a slightly smaller size. Position C generally corresponds to the elastic limit of the medical device. It is then relaxed to a slightly larger size, the same size as originally selected at position B, with the much-reduced stress and loading at position D.

An additional advantage of the present invention is that, upon deployment and expansion to working size, the hysteresis loop of FIG. 2 causes the stent to regain its full hoop strength.

Among many kinds of modifications and features that may be provided with the delivery system of the present invention are (i) a relatively small profile, (ii) several radiopaque marker bands indicating the positions of certain components, (iii) flexibility, (iv) minimization of any sharp edges when advancing or withdrawing the catheter system or when retracting the outer sheath, (v) optimized longitudinal force transmission, (vi) materials selected for performance, and (vii) affirmative release of the self-expanding stent when the sheath is retracted. The delivery system preferably also provides stent position retention, as well as stent protection, during insertion of the catheter. Like many catheter systems, a delivery system for a resilient medical device is often used with a flexible guidewire. The guidewire is often metal, and is slidably inserted along the desired body passage. The catheter system is then advanced over the guidewire by “back-loading” or inserting the proximal end of the guidewire into a distal guidewire port leading to a guidewire lumen defined by the catheter system.

Many catheter systems define guidewire lumens that extend along all or almost all the length of the catheter. These catheter systems are described as “over-the-wire” catheters, in that the guidewires remains inside a catheter lumen throughout the length of the catheter. Over-the-wire catheter systems provide several advantages, including improved trackability, preventing prolapse of the guidewire, the ability to flush the guidewire lumen while the catheter is in the patient, and the capability of easily removing and exchanging the guidewire while retaining the catheter in a desired position in the patient.

Over-the-wire delivery systems may include an inner tubular body defining a guidewire lumen and providing a spine around which a compressed stent is mounted, as well as an outer sheath for containing the stent until it is pulled proximally to release the stent.

In some circumstances it may be desirable to provide a “rapid-exchange” catheter system, which offers the ability to easily remove and exchange the catheter while retaining the guidewire in a desired position within the patient, using rapid-exchange features generally known in the art.

These and various other objects, advantages and features of the invention will become apparent from the following description and claims, when considered in conjunction with the appended drawings.

### BRIEF DESCRIPTION OF THE DRAWINGS

FIG. 1 is an external perspective view of a medical device delivery system, arranged according to the principles of the present invention;

FIG. 2 is a diagrammatic view of a resilient medical device according to the principles of the present invention, illustrating a sample stress-strain curve of a possible material for the medical device;

FIG. 3 is a diagrammatic view of a medical device, illustrating a sample stress-strain curve for a material such as steel;

FIGS. 4 and 6 are elevation views of medical devices;

FIG. 5 is an elevation view of the medical device of FIG. 4 in an expanded shape;

FIG. 7 is an external perspective view of a medical device;

FIG. 8 is a diagrammatic plan view of the medical device of FIG. 6;

FIG. 9 is a diagrammatic plan view of a medical device;

FIG. 10 is an elevation view of a vascular filter according to the present invention; and

FIG. 11 is a perspective view of a vascular graft according to the present invention.

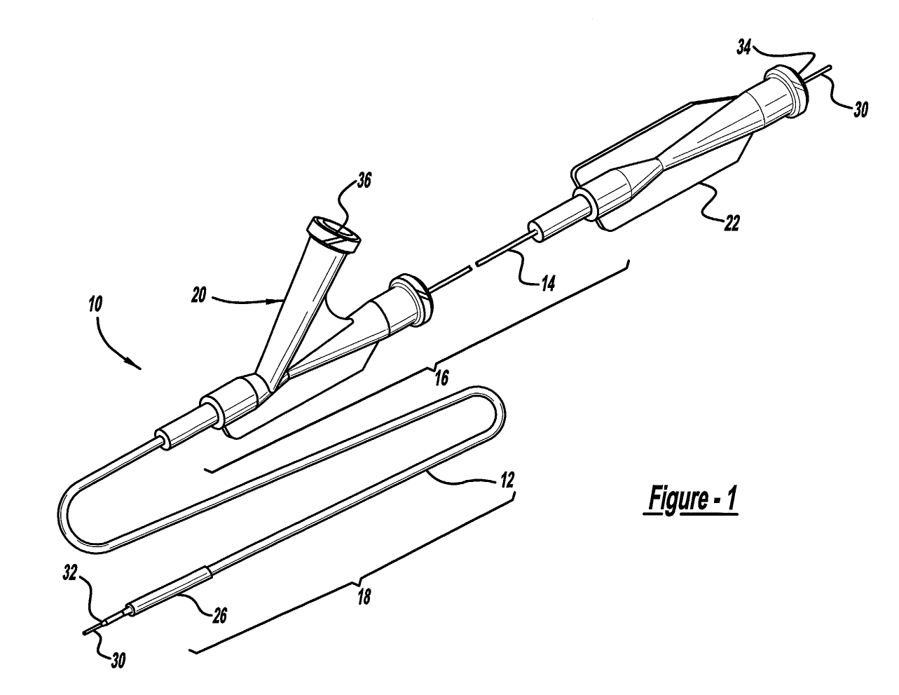

### DETAILED DESCRIPTION OF THE PREFERRED EMBODIMENT

The following description of the preferred embodiments of the present invention is merely illustrative in nature, and as such it does not limit in any way the present invention, its application, or uses. Numerous modifications may be made by those skilled in the art without departing from the true spirit and scope of the invention.

Referring to the drawings, a medical device delivery system is depicted, with one of the preferred embodiments of the present invention being shown generally at 10. The illustrated stent delivery catheter system 10 of course depicts only one of many different medical device delivery system designs within the scope of the present invention. For clarity and convenience, the present detailed description will only describe delivery systems for stents. However, the novel technique of the present invention may of course also be used with a variety of other resilient medical devices. Such other medical devices may include for example vascular filters, vascular grafts, valves, clips such as anastomosis and aneurysm clips, coaxial tube devices such as endoscopes and optical fiber guides, and orthopedic devices such as bone anchors and surgical devices.

In the illustrated embodiment, the medical device delivery system 10 shown in the drawings includes outer and inner shaft bodies 12 and 14 each having proximal and distal ends 16 and 18, first and second proximal hubs 20 and 22 attached to the outer and inner shaft bodies 12 and 14 respectively, and a resilient stent 24.

The outer shaft body 12 is preferably a relatively long and flexible tube, through which the inner shaft body 14 is received and can move in a proximal or distal direction. The outer shaft body 12 may preferably have at its distal end a tubular cartridge 26, which can receive and hold the stent 24 in compression.

The inner shaft body 18 is preferably relatively long and flexible as well. The inner shaft body 18 may be a solid member, though it is also preferably a tube, and may be made of metal hypotube material.

The present stent delivery system designs preferably also preferably provide a guidewire lumen for slidably receiving a long flexible guidewire 30, extending from a distal port 32 defined by a distal end 18 of the inner shaft body 14 to a proximal port 34 defined at the proximal end 16 of the second hub 22.

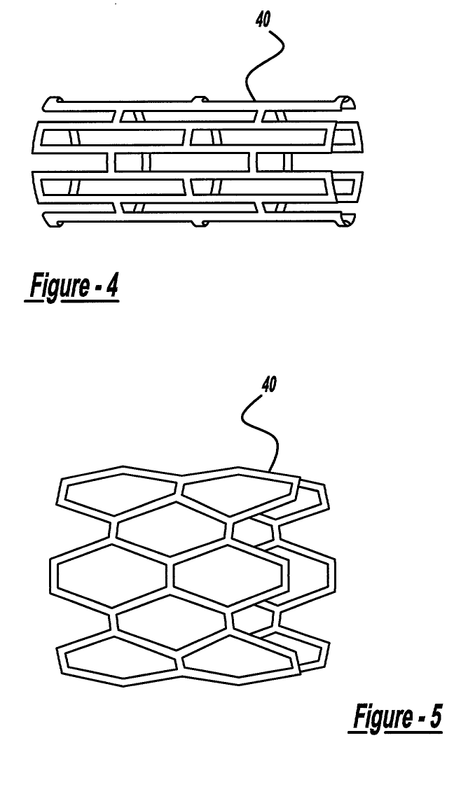

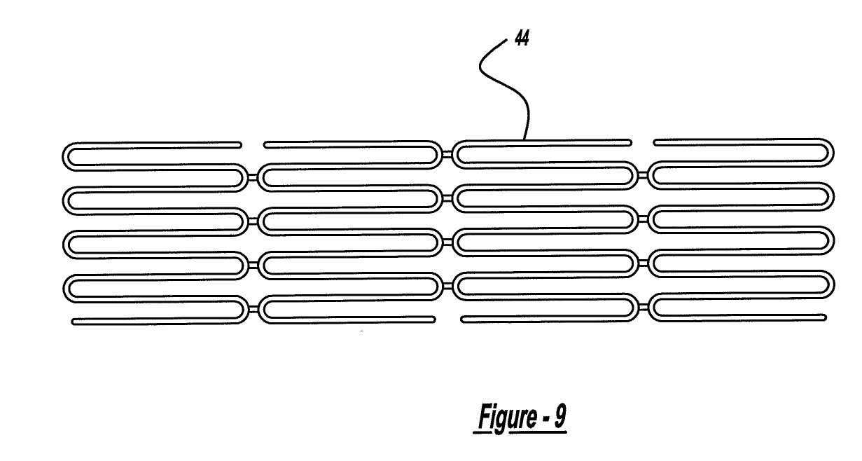

The stent 24 is preferably of the self-expanding type, usually a tubular structure resiliently tending to expand from a first delivery diameter to a second deployed diameter. There are of course a wide variety of stent designs which are acceptable for the stent delivery system of the present invention. One possible design is a stent made of metal wire that is bent and wound into the desired shape, of a type similar to that shown in FIG. 9. A second possible design is a stent made of a cylindrical tube that is selectively cut to form a flexible tubular lattice, of a type similar to those shown in FIGS. 4-8. Stent 24 is preferably made of a resilient material such as nitinol.

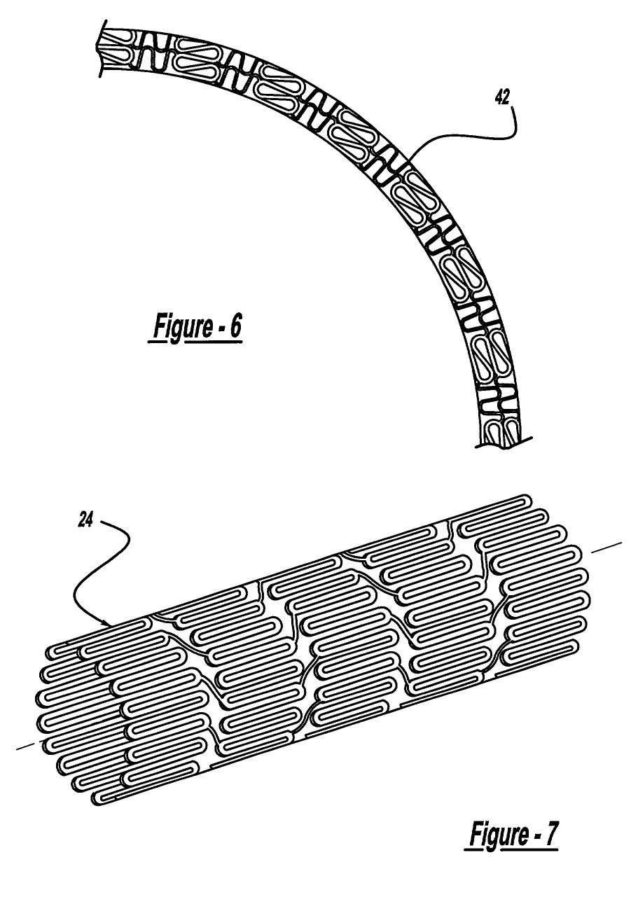

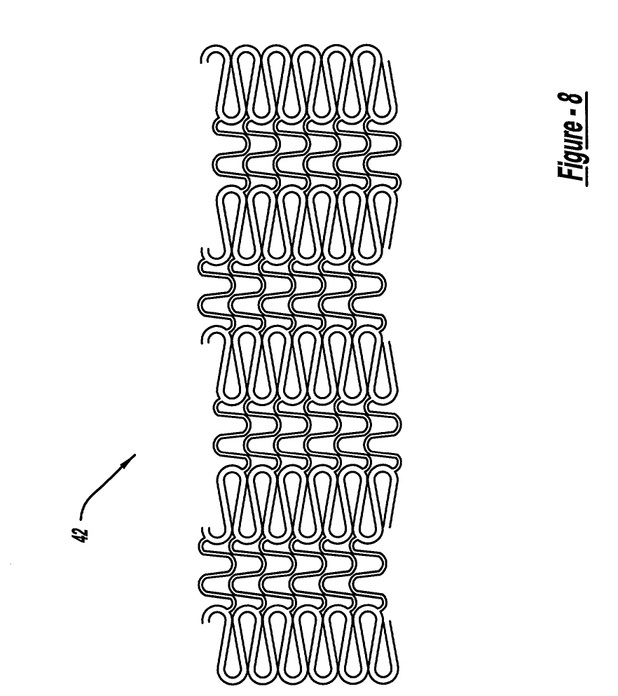

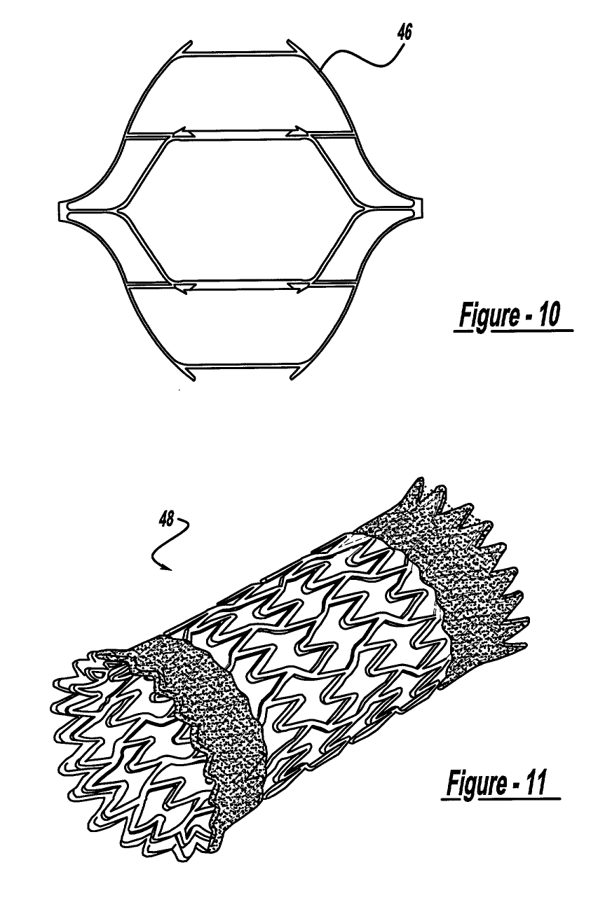

The techniques of the present invention may be used with stents of various suitable designs, including the alternate designs of stent 40 in FIGS. 4-5, stent 42 in FIGS. 6 and 8, and stent 44 in FIG. 9. In addition, the present invention may be used with a variety of other types of medical devices, including for example the resilient vascular filter 46 in FIG. 10, and the covered sheet or stent graft in FIG. 11.

The present stent delivery system may have a configuration referred to as “over-the-wire” because the proximal guidewire port is located in the proximal hub, such that the guidewire is enclosed within the catheter shaft for the full length of the catheter. In contrast, the present stent delivery system may have “a rapid exchange” design, which provides a proximal guidewire port at an intermediate position, and may have the capability of removing and exchanging the catheter system without removing the guidewire 30.

The first proximal hub 20 is affixed to the proximal end of the outer shaft body 12. As with all mechanical interfaces of the present stent delivery system, those of average skill in the art will be well aware of various methods of coupling components, including adhesives, heat-welding, interference close fit, snap-lock, a combination of known methods, etc. The first hub 20 also preferably has a flush port 36 for receiving liquids such as saline solution, radiopaque contrast or therapeutic drugs and communication the liquid along a lumen defined by the outer shaft body 12.

The inner and outer shaft bodies may of course be made of various materials, including stainless steel, nitinol, a polymer or any other biocompatible material with the desired physical properties, including a relatively high column strength, flexibility, etc. At its proximal end, the inner shaft body 14 is connected to the second hub 22, which provides a maneuvering handle for a physician to operate the device and delivery system.

A self-expanding stent 24 of suitable type or configuration is preferably provided with the present stent delivery system, such as the SMART stent, available from Cordis Corporation, Miami, Fla. Various kinds and types of self-expanding stents are acceptable for use in the present invention, as well as new stents which may be developed in the future that are compatible with the present stent delivery system. When deployed in a body passageway of a patient, the stent is preferably designed to press radially outward to hold the body passage open.

The stent cartridge 26 is affixed to the distal end of the outer body 30, and surrounds the compressed stent 24 during delivery to the desired site. When the stent 24 is in position, the cartridge 28 is withdrawn in the proximal direction by holding the second hub stationary and pulling on the first hub 20, to expose and deploy the stent 24. The stent cartridge 26 is permanently mounted about the inner body 26, yet is able to slide a short distance longitudinally back and forth relative to the inner body 26. A stop (not shown) is preferably affixed to the inner body 14 adjacent to the stent 24 proximal end, to inhibit the stent 24 from shifting position as the outer body 12 and stent cartridge 26 are withdrawn.

The guidewire 30 and stent delivery system 10 may be advanced or withdrawn independently, or the catheter 12 may be guided along a path selected with the guidewire 30.

As explained above, the present invention provides a novel technique of the intentionally “over-compressing” the stent or other medical device to a size slightly smaller than eventually desired, using much greater pressure, and then “relaxing” the stent by allowing it to expand slightly to the desired initial size. The result of this over-compress and relax process is to greatly reduce the stress in, and the expansive force of the stent against, the delivery system. The delivery system can accordingly be designed with a smaller distal profile, thinner components, and have a longer shelf life and greater flexibility, because it needs to support and contain less stress or expansive force.

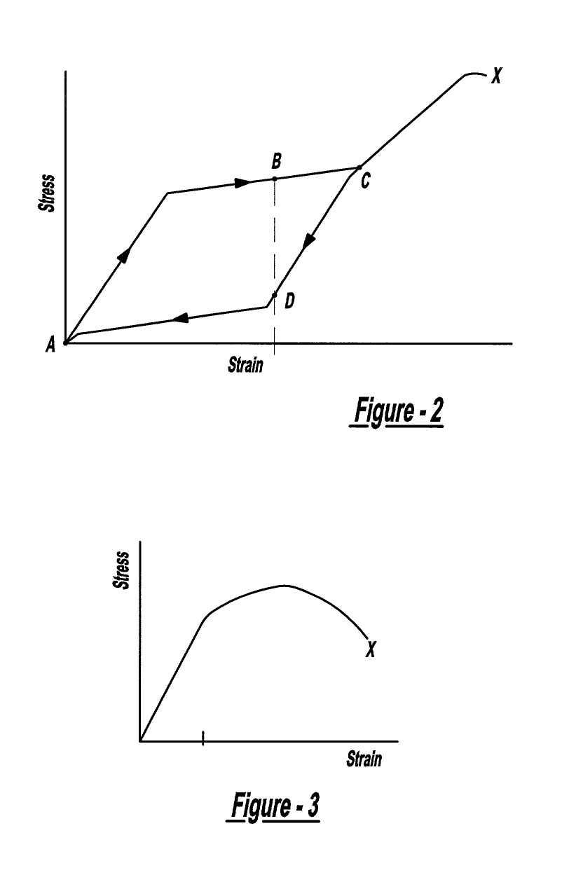

The reason for this greatly reduced stress can be understood graphically by referring to FIG. 2, which shows a sample diagrammatic stress-strain curve having a hysteresis loop. When the stent or other resilient medical device is first made, it has an initial expanded and relaxed size, at position A in FIG. 2. When it is compressed to the smaller size necessary to fit in the delivery system, at approximately position B, it presses outward with high stress. Prior devices and delivery systems carry this relatively higher stress level throughout packaging, sterilization, storage, shipping, and delivery into the patient.

According to the present invention, the device is preferably over-compressed approximately to position C in FIG. 3, at slightly higher stress and compressed to a slightly smaller size. Position C corresponds to the elastic limit of the stent or other medical device. It is then relaxed to a slightly larger size, the same size as originally selected at position B, with the much-reduced stress and loading at position D.

An additional advantage of the present invention is that, upon deployment and expansion to working size, the hysteresis loop of FIG. 3 causes the stent to regain its full hoop strength.

Among many kinds of modifications and features that may be provided with the delivery system of the present invention are (i) a relatively small profile, (ii) several radiopaque marker bands indicating the positions of certain components, (iii) flexibility, (iv) minimization of any sharp edges when advancing or withdrawing the catheter system or when retracting the outer sheath, (v) optimized longitudinal force transmission, (vi) materials selected for performance, and (vii) affirmative release of the self-expanding stent when the sheath is retracted. The delivery system preferably also provides stent position retention, as well as stent protection, during insertion of the catheter.

Catheter designs tend toward very small profiles at their distal tips, with the understanding that a small profile might assist in crossing a narrow lesion. This profile is simply defined by the outer diameter of the inner tube at its distal end, which may even be drawn down to an even smaller diameter. Again, one of the advantages of the present invention is that the profile can be further minimized, yet while maintaining acceptable strength and performance because it is a subject to less stress.

Various different materials may be used for the various components of a stent delivery system according to the present invention. Most of the catheter components should preferably be made of materials having acceptable properties including biocompatibility, pull strength, longitudinal or column strength, and bending flexibility. Some of the preferred materials may include various plastics, referred to as polymers, including nylon, polyethylenes, polyurethanes, or PET.

For example, the proximal inner shaft body 18, and any guidewire used with the present stent delivery system, is preferably made of metal such as stainless steel, while the distal inner body, outer body, cartridge, tapered tip, and first and second hubs may be of polymers.

In the particular preferred embodiment shown in the drawings, a specific set of materials has been selected. The inner body 12 and the cartridge 26 are preferably a sandwich of multiple polymer layers, possibly with a reinforcing metal coil or braid. A lubricious coating, for example PTFE, may be added to the inner guidewire lumen of the inner body 14.

Likewise, outer body 12 is preferably also a co-extrusion or layered polymer construction, with polymer layers which may for example be nylon, polyethylene, polyurethane, PEEK, or a block copolymer thereof.

The inner body 14 preferably has a radiopaque marker (not shown) adjacent to the proximal end of the stent 24, which preferably defines an outwardly extending surface for resisting motion of the stent 24 as the stent cartridge 26 is retracted. Inner body 14 may also have a second radiopaque marker (not shown) adjacent to the distal end of the stent 24. Radiopaque markers are visible with fluoroscopy during medical catheter procedures, enabling a physician to see the stent ends with x-rays. Various radiopaque materials are available for the markers, including gold, iridium, and platinum.

In operation of the stent delivery system 10, the stent delivery system 10 may be first back-loaded onto a guidewire 30 already positioned along the body passage to the desired site, by inserting a proximal end of the guidewire 30 into distal guidewire port 32. The stent delivery system 10 may be inserted percutaneously through an outer guiding catheter (not shown) and along a guidewire already positioned along the body passage to the desired site. The stent delivery system 10 is then advanced along the guidewire 30, until the stent 24 covered by the cartridge 26 is positioned within the lesion (not shown).

The sheath or cartridge 26 is then partially retracted, to uncover the stent 24. The stent delivery system 10 is removed from the patient's body after full deployment, leaving the stent 24 implanted at the desired site.

Catheter manufacturing techniques are generally known in the art, including extrusion and co-extrusion, coating, adhesives, and molding. The scope of the present invention encompasses the full extent of the claims, regardless of specific numbers, materials, or other details present in this description of the preferred embodiment.

Preferably, the catheter hubs 20 and 22 are injection molded of any suitable material. The inner and outer shaft tubes 14 and 12 are preferably made of a polymer such as nylon, the material stiffness of which may be selected as appropriate.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A method of assembling a resilient medical device and a delivery system to reduce stress in the medical device, comprising:
providing a resilient medical device having the property of a stress-strain curve including a hysteresis loop;
providing a catheter delivery system having a tubular body defining an inner diameter;
compressing the resilient medical device to a size smaller than the tubular body inner diameter, thereby increasing the stress in the resilient medical device to an initial amount;
inserting the resilient medical device into the tubular body, and allowing the resilient medical device to resiliently expand inside the tubular body to a size corresponding to the tubular body inner diameter, thereby greatly reducing the stress in the resilient medical device from the initial amount to a reduced amount.

2. The method of claim 1, wherein stress-strain curve of the resilient medical device has a compressing path and an expanding path which meet at a point of convergence, the compressing path exhibiting a greater stress for a given strain than the expanding path, and wherein the medical device exhibits only a single path at strains greater than the convergence point; such that the initial amount is a point on the compressing path of the hysteresis loop and has a stress greater than the reduced amount, and the reduced amount is a point on the expanding path of the hysteresis loop.

3. The method of claim 1, wherein the stress of the medical device at the reduced amount is half or less than the initial amount of stress.

4. The method of claim 1, wherein the medical device is a self-expanding stent.

5. The method of claim 1, wherein the medical device is a vascular filter.

6. The method of claim 1, wherein the medical device is formed of nitinol.

7. The method of claim 1, wherein the resilient medical device is a vascular graft.

8. The method claim 4, wherein the catheter delivery system has a reduced outer profile diameter, to support the reduced amount of stress in the medical device.

9. The method of claim 1, wherein the catheter delivery system further comprises an outer tubular member having a thinner wall thickness to support the reduced amount of stress than for a corresponding greater stress at an equivalent strain on the compressing path of the hysteresis loop.

10. The method of claim 1, wherein the initial amount of stress in the resilient medical device corresponds to the elastic limit of the medical device material.

11. The method of claim 2, wherein the point of convergence is the elastic limit of the medical device material.

12. The method of claim 2, wherein the strain of the medical device is equal at the initial and reduced amounts of stress.

13. A catheter delivery system and a resilient medical device for treating a patient, comprising:
a resilient medical device having the property of a stress-strain curve including a hysteresis loop;
a catheter delivery system having a proximal and distal end, and including an outer tubular member;
wherein the medical device is enclosed within and constrained by a portion of the outer tubular member;
wherein the stress-strain curve of the medical device follows a compressing path of relatively greater stress than an expanding path of relatively lesser stress; wherein the compressing and expanding paths meet at an elastic limit;
such that the medical device when constrained to a pre-delivery strain by the outer tubular member in a pre-delivery configuration has a stress amount corresponding to a point on the expanding path of relatively less stress.

14. The delivery system and medical device of claim 13, wherein the stress of the medical device at the reduced amount is half or less than the initial amount of stress.

15. The method of claim 1, wherein the medical device is a self-expanding stent.

16. The method of claim 1, wherein the medical device is a vascular filter.

17. The method of claim 1, wherein the medical device is formed of nitinol.

18. The delivery system and medical device of claim 13, wherein the medical device follows the expanding path upon delivery and release from the catheter delivery system, at least a portion of the expanding path having a steep slope, such that the medical device recovers substantially all of its resilient strength.

19. The delivery system and medical device of claim 13, wherein the combined delivery system and medical device has greater flexibility, resulting from the reduced stress in the medical device.

20. The delivery system and medical device of claim 13, wherein the combined delivery system and medical device has greater trackability, resulting from the reduced stress in the medical device.
